(19) United States
(12) Patent Application Publication
(10) Pub. No.: US 2005/0033197 A1
(43) Pub. Date: Feb. 10, 2005

(54) Apparatus for fluid transport and related method thereof

(76) Inventor: Patrick S Cottler, Charlottesville, VA (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/497,815

(22) PCT Filed: Dec. 6, 2002
(86) PCT No.: PCT/US02/39251

Related U.S. Application Data
(60) Provisional application No. 60/337,310, filed on Dec. 6, 2001.
(60) Provisional application No. 60/340,516, filed on Dec. 13, 2001.

(51) Int. Cl. A61B 5/00; B65D 81/00; A61M 1/00; A61M 31/00

(52) U.S. Cl. 600/573; 604/35; 604/506

(57) ABSTRACT

An apparatus and method capable of percutaneous or direct removal of blood and other body fluids, or infusion of medicinal, therapeutic, bioactive mixtures. The fluid transport apparatus includes a fluid collection chamber (20); cutting devices (50) attached to the collection chamber to penetrate the skin of the patient (2); and a vacuum supply (61) in communication with the fluid collection chamber (20); whereby the vacuum supply (61) providing a vacuum pressure to remove congested fluid from the patient through the cutting devices (50) into the collection chamber (20). Further, the apparatus may provides conduits (28) disposed on the collection chamber (20) that are juxtaposed or concentric with the cutting devices (50), whereby vacuum pressure to removes the congested fluid from the patient (2) through the conduit (28) into the collection chamber (20).

### CROSS-REFERENCES TO RELATED APPLICATIONS

The present invention claims priority from U.S. Provisional Patent Application Ser. No. 60/337,310 filed Dec. 6, 2001, entitled “A Vacuum Apparatus for Fluid Transport and Related Method thereof,” and 60/340,516 filed Dec. 13, 2001, entitled “A Vacuum Apparatus for Fluid Transport and Related Method thereof,” the entire disclosures of both are hereby incorporated by reference herein in their entirety.

The present application is related to PCT International Application No. PCT/US00/16880, filed Jun. 19, 2000, by Cottler et al., entitled “An Apparatus for Fluid Transport and Related Method Thereof,” of which is hereby incorporated by reference herein in its entirety.

### FIELD OF THE INVENTION

The present invention relates generally to an apparatus and method thereof for withdrawing fluids, and more particularly to an apparatus and method capable of percutaneous or direct removal of blood and other body fluids, as well as infusion of materials into the body.

### BACKGROUND OF THE INVENTION

Reconstructive and plastic surgery often involves the transfer of tissue to deep defects, where skin grafts would not be beneficial. During such transfer, the harvested tissue is without blood flow. If the blood flow is not restored quickly, due to microvascular complications, part or all of the tissue may become necrotic and will fail. One such complication, venous congestion, involves inadequate venous drainage with a patent arterial inflow, and is due to such things as tissue edema, venous thrombosis, leukocyte aggregation, or the fact that in some cases, microvascular reconnections of the venules are not surgically possible.

By way of background, skin flaps are a common feature utilized by plastic surgeons to reconstruct defects and to cover deep wounds in which a skin graft or replacement is not feasible because a patent vascular bed is absent. A skin flap is a multi-layered tissue that includes dermis, epidermis, subcutaneous tissue, fasciocutaneous, myocutaneous, osseocutaneous, and sometimes muscle tissue, sensory tissue, and possibly underlying adipose tissue, which is based on its own microvascular network. There are many thousands of cases each year that require the use of skin flaps to some degree during a medical procedure whereby these procedures can cost thousands of dollars. Reconnection of arteries to establish adequate arterial flow is vital for the ultimate success of these microsurgical procedures.

Venous congestion is also a clinical indication seen in surgical tissue replantations. Such replantations include the reattachment of traumatically amputated fingers, toes, ears, nose, etc.

Nonetheless, skin flaps and replants with proper arterial flow can still have compromised venous outflow, known as venous congestion, for a variety of reasons. Venous insufficiency has adverse effects, and leads to a majority of the failures in replantation surgeries. Venous congestion is a clinical problem in which extensive effort has been spent in attempts to alleviate or prevent its onset.

As a result, fluid transport techniques such as medicinal leeches are used as a treatment option in these cases to promote nutritive blood flow. The medicinal leech is currently used to initiate blood flow and reduce tissue swelling in skin flaps or replanted tissues, and to promote nutritive blood flow. The medicinal leech attaches to its host utilizing three semicircular jaws containing approximately sixty (60) pairs of cutting teeth to create a “Y” shaped incision of approximately 4-5 mm in diameter. Once attached, the bioactive saliva (including anticoagulants, antiseptics, anesthetics, vasodilators, etc.) is secreted between each pair of teeth, and the nervous system stimulates the pharynx to pump peristaltically, creating a negative pressure, which aids in driving blood flow into the leech. Leeches will feed until stretch receptors are stimulated by distention in the body. It has been reported that feeding on large mammals, if allowed to proceed undisturbed, will last from 20 to 60 minutes, in which time the leech will ingest 5 to 15 ml, which is up to ten times the initial body weight. Once detached, the bite wound will continue to bleed, which is thought to be an important portion of the therapy. The wound will “ooze” up to 50 ml more in the 24 to 48 hours after feeding. In order for this secondary part of the treatment to be effective, the wound must be continuously cleared of thromboses that form on the patient's skin surface.

The use of leeches establishes a zero or negative pressure outlet for several vessels in the congested area. With the outlet, flow can resume at a basal level, supplying minimal necessary nutrients to the flap. If the flap can survive long enough due to this artificial flow, venous congestion can be alleviated by resolving thromboses or vessel reconnections can form, leading to survival of the tissue.

As stated previously, one of the major factors in a successful tissue transplantation or replantation is the reestablishment of blood flow. The most common cause of flap failure is venous insufficiency, which can be treated with the use of medicinal leeches. However, the use of leeches carries the risk of infection and offers poor flow control. Other potential problems for the patient that can arise with the use of the medicinal leech range from psychological problems, such as fear and disgust, to pharmacological problems in nature. Once a patient is comfortable with the procedure, there are other potential problems that can arise. Leeches rely on bacterium for the digestion of the ingested blood, due to the lack of proteolytic enzymes within the gut. One such bacterium is Aeromonas hydrophilia, a gram-negative rod, which has led to septicaemia, pneumonia and gastroenteritis in humans. These bacteria may be ejected into the patient and cause infection. Infection rates of up to 20 percent have been linked to A. hydrophilia from leeches. Some patients can experience anaphylaxis and allergic responses to the bioactive saliva of leeches, while others could exhibit excessive scarring from the bite wound site. Continued leech use and persistent bleeding from the wound can result in a significant loss in blood volume. Hemoglobin levels can drop by 1 to 2 gm percent over a five day treatment due to the amount of blood lost, thus requiring a blood transfusion. Finally, if the leech is not monitored, it may wander to a more perfused region of the body to initiate feeding, rendering the treatment useless. Leeches are a widely used clinical tool today as evidenced by Biopharm Ltd. and Leeches USA Ltd. typically supplying over 100,000 leeches per year. Due to these potential shortcomings of medicinal leeches, alternative methods to leech treatments would be very important to the future of microsurgery.

An alternative approach to medicinal leeches is a mechanical leech disclosed by Smoot et al. in an article entitled “Mechanical Leech Therapy to Relieve Venous Congestion,”Journal of Reconstructive Microsurgery, Vol. 11, No. 1, January (1995) of which is hereby incorporated by reference herein. It appears that the Smoot device is arranged with an elongated suction chamber with an inflow port for heparinized saline and an outflow port for continuous suction. In operating mode, the Smoot device is placed over a biopsy wound measuring 4 mm in diameter and suction is adjusted to achieve a negative pressure.

While the Smoot device appears to overcome some of the psychological and pharmacological failings of traditional medicinal leeching, as it attempts to replace a natural leech, it nevertheless has its own shortcomings. In this regard one shortcoming of the Smoot device is that it is not self-contained. Another major shortcoming relative to the present invention is that the suction pressure of the Smoot device is extremely large (i.e., −80 mmHg), and can not be varied cyclically. Cyclic variations are useful to maintain good flow and prevent blood clotting and/or clogging of channels that communicate fluid from the body to the collection reservoir. Such a large negative pressure, as required by the Smoot device, could collapse blood vessels and compact tissue, leading to clogging of transport channels. Another shortcoming of the Smoot device is that the outlet size of the biopsy wound is much greater than the required insertion of the present invention and could even be considered a wound that is detrimental to the patient. A further shortcoming of the Smoot device is that the flow of heparinized saline leads to the susceptibility of spillage. An additional shortcoming of the Smoot device is that the device itself is not contained within a self-powered unit. The Smoot device is also unlikely to work clinically on a venous congested tissue. A final shortcoming of the Smoot device is that a punch biopsy is required in the flesh of the subject prior to the “leech” being used, and consequently an increased risk of contamination. Clinicians have previously used skin excisional wounds (large area wounds) to treat seriously coagulated skin flaps. So the Smoot device essentially adjusts suction to a large area wound and would not be effective for venous congested flaps.

There is therefore a need in the art for an effective mechanical leech apparatus for percutaneous or direct removal of blood and other body fluids which does not suffer the disadvantages associated with a medicinal leech and/or conventional mechanical leeching methods.

### SUMMARY OF THE INVENTION

A novel approach for withdrawing or infusing fluids, and more particularly to an apparatus and method capable of percutaneous or direct removal of blood and other body fluids, or infusion of medicinal, therapeutic, bioactive mixtures. In one aspect, the present invention features a fluid transport apparatus for withdrawing fluid from a target or patient, the fluid transport apparatus comprising: at least one fluid collection chamber; at least one cutting device attached to the collection chamber to penetrate the skin of the patient; and a vacuum supply in communication with the fluid collection chamber; the vacuum supply providing a vacuum pressure to remove congested fluid from the patient through the at least one cutting device into the collection chamber.

In another aspect, the present invention features a fluid transport apparatus for withdrawing fluid from a target or patient, the fluid transport apparatus comprising: at least one fluid collection chamber; at least one cutting device disposed on the collection chamber to penetrate the skin of the patient to form at least one aperture in the skin; at least one conduit disposed on the collection chamber, the at least one cutting device having at least a portion disposed inside the at least one conduit; and a vacuum supply in communication with the fluid collection chamber; the vacuum supply providing a vacuum pressure to remove congested fluid from the patient through the at least one conduit into said collection chamber. In one embodiment, the cutting device has a protrusion position whereby its distal end is located beyond the distal end of the conduit. In another embodiment, the cutting device has a retraction position whereby its distal end is even with or below the distal end of the conduit, wherein the cutting device is adapted to move from the protrusion position to the retraction position. Further yet, another embodiment the cutting device has a retraction position whereby its distal end is even with or below the distal end of said at least one conduit, wherein the cutting device is adapted to move from the retracted position to the protrusion position and then to a retraction position, and repeat partially or completely a predetermined number of times.

Still yet, the present invention features a fluid transport apparatus for withdrawing fluid from a target or patient, the fluid transport apparatus comprising: at least one fluid collection chamber; at least one cutting device disposed on the collection chamber to penetrate the skin of the patient to form at least one aperture in the skin; at least one conduit disposed on the collection chamber and juxtaposed with the at least one cutting device; and a vacuum supply in communication with the fluid collection chamber, wherein the vacuum supply providing a vacuum pressure to remove congested fluid from the patient through the at least one conduit into the collection chamber.

Yet again, the present invention features a fluid transport apparatus and method for withdrawing fluid from a patient, whereby the patient has at least one target passage extending therein. The fluid transport apparatus comprising: at least one fluid collection chamber; at least one aperture (and/or conduit) in communication with the collection chamber, the at least one aperture (and/or conduit) is adapted to be aligned with the at least one of the target passage of the patient; and a vacuum supply in communication with the fluid collection chamber; the vacuum supply providing a vacuum pressure to remove congested fluid from the patient via the at least one target passage and through the at least one aperture (and/or conduit) into the collection chamber.

Additionally, another aspect of the invention provides a method of transporting fluid from a patient using a fluid transport apparatus, the method comprising: inserting at least one cutting device into the patient wherein the at least one cutting device is disposed on the fluid collection chamber; providing a vacuum supply in communication with the fluid collection chamber; the vacuum supply providing a vacuum pressure to remove congested fluid from the patient through the at least one cutting device into the collection chamber.

Moreover, another aspect of the invention provides a method of transporting fluid from a patient using a fluid transport apparatus, the fluid transport apparatus comprising: inserting at least one cutting device into the patient wherein the at least one cutting device is disposed on the collection chamber; providing at least one conduit disposed on the collection chamber, the at least one cutting device having at least a portion disposed inside the at least one conduit; and providing a vacuum supply in communication with the fluid collection chamber; the vacuum supply providing a vacuum pressure to remove congested fluid from the patient through the at least one conduit into the collection chamber.

Finally, another aspect of the invention provides a method of transporting fluid from a patient using a fluid transport apparatus, the fluid transport apparatus comprising: inserting at least one cutting device into the patient wherein the at least one cutting device is disposed on the collection chamber; providing at least one conduit disposed on the collection chamber, the at least one cutting device being located in close proximity of the at least one conduit; and providing a vacuum supply in communication with the fluid collection chamber; the vacuum supply providing a vacuum pressure to remove congested fluid from the patient through the at least one conduit into the collection chamber. These and other objects, along with advantages and features of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings in which:

FIGS. 1-2 are schematic illustrations showing a fluid transport apparatus for withdrawing fluid from a body as part of an embodiment of the present invention.

FIGS. 3A-3B show a schematic plan view and side view illustration, respectively, of a collection chamber having a curve surface or communication surface.

FIG. 4 show a schematic side view illustration, respectively, of a collection chamber having a curve surface or communication surface.

FIGS. 5A-5B schematically show a collection chamber and an infusion reservoir having infusion channels as part of the fluid transport apparatus.

FIGS. 6 and 8 are side view schematic illustrations showing a fluid transport apparatus for piercing the skin with cutting devices and withdrawing fluid from a body through conduits.

FIGS. 7 and 9 are enlarged partial views of FIGS. 6 and 8, respectively, showing a conduit and cutting device of the fluid transport apparatus.

FIGS. 10A-10B show schematic illustrations of a top view and a bottom view, respectively, of the apparatus as shown in FIGS. 6 and 8.

FIGS. 10C-10D show schematic illustrations of a top view and a bottom view, respectively, wherein the cutting devices and hollow conduits are in juxtaposed relation, i.e., near proximity next to one another.

FIGS. 11-13 show schematic illustrations of examples of embodiments of the present invention apparatus that are intended for illustrative purposes only and are not intended to be limiting in any manner.

### DETAILED DESCRIPTION OF THE INVENTION

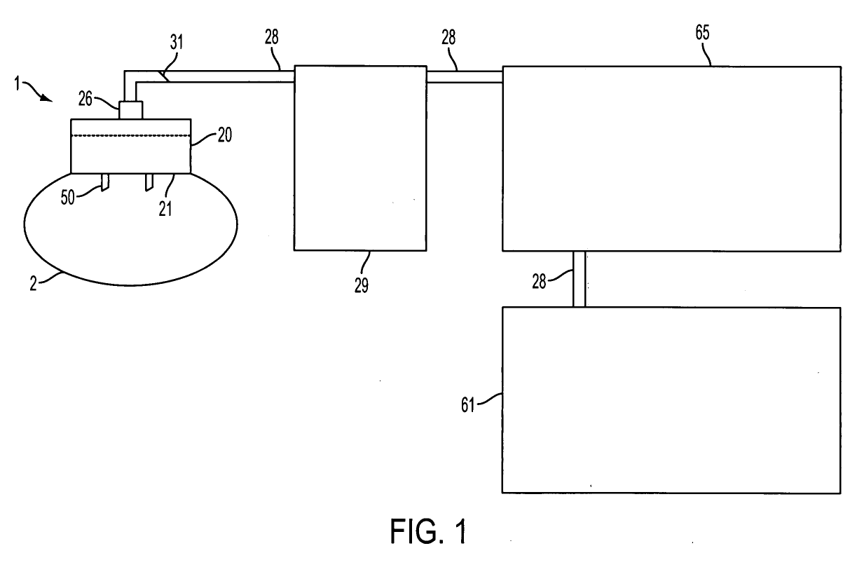

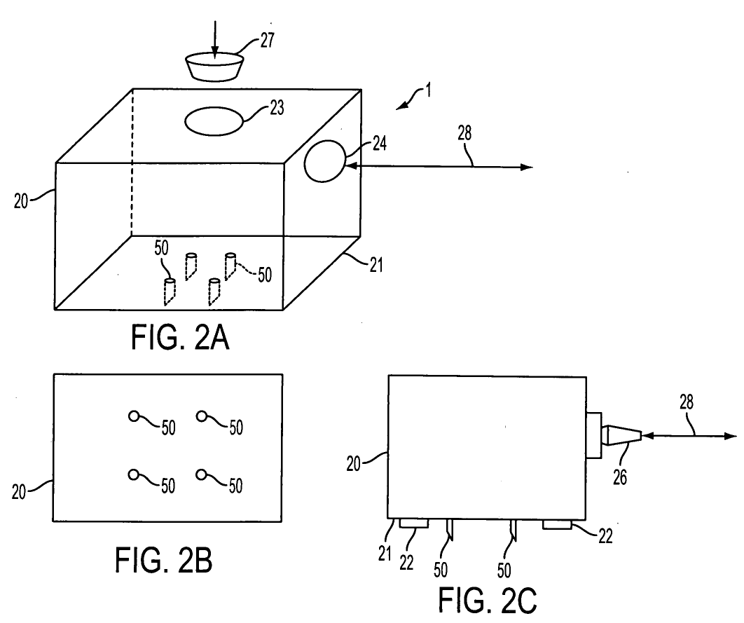

Turning now to the drawings, FIGS. 1-2 are schematic illustrations showing a fluid transport apparatus 1 for withdrawing fluid from a body 2 as part of an embodiment of the present invention. For discussion purposes, the fluid transport apparatus 1 will be described in the context of an apparatus and method capable of providing a percutaneous or direct removal of blood and other body fluids from the body or target 2, but is not limited thereto. The present invention apparatus and related method thereof can be utilized in but not limited to plastic surgery clinics for patients that experience venous congestion in tissues following a skin flap transfer and replanted digits or other extremities, as well as patients that develop deep venous thromboses. It should be appreciated that the present invention apparatus and method may be utilized for general wound healing such as for example, to relieve pressures or provide removal of accumulated fluid. The fluid transport apparatus 1 has a fluid collection chamber 20 that has attached thereto cutting devices 50. The cutting devices 50 are used to make incisions through the wall or skin of the body or target 2 by inserting the cutting device or devices 50 through the wall of the body for automatically removing blood or other tissue fluids from the skin or other tissues. In an embodiment, the depth of penetration of the cutting devices 50 is about 0.1 mm to about 15 rpm in depth, and could be lesser or greater in other tissue applications. Merely by way of example, the cutting devices 50 may be hypodermic needles, or equivalent known construction, having an inner diameter in the range of about 100 μm to about 8,000 μm. Alternatively, the inner diameter may have a range of about 200 μm to about 840 μm. Also, the cutting device may be a tubular structure, conduit, tube or the like.

Still referring to FIGS. 1-2, the present invention apparatus and method involves the collection chamber 20 that contains a collection reservoir for removed congested fluid and has a communicating surface 21 to interface with the patient's congested tissue 2. Volume of the collection reservoir is envisioned to be on the order of about 1-100 ml, but could vary depending on the application, e.g. about 5-10 ml or 2-35 ml. This chamber 20 (receptacle, well, enclosure, repository, holder, or the like) will then be in communication with a vacuum source 61 that could be regulated by some sort of regulator mechanism 65. The chamber 20 can be a single or multi-compartmental that could optionally have valves 31 that would maintain flow from the patient into the collection reservoir. With the variety of potential body parts (finger tips, toes, ears, legs, variety of sizes and placements of skin flaps, etc.) that might need treatment this device could be flexible or have multiple shapes, curvatures, and sizes that would all be appropriate for the present invention apparatus. The collection reservoir or chamber 20 will be manufactured with a material that will give the most advantageous material properties to perform the treatment effectively and adhere to the contours of the patient 2. For example, such materials may include, but not limited to the following: low gauge metal, plastic, rubber, and other polymers.

Other variations to the present invention apparatus could include but not limited thereto, size of the compartments such as the collection chamber 20 and infusion chamber 33, footprint at the patient interface surface or communication surface 21, resting curvature of patient interface surface or communication surface 21, or mechanical rigidity of any compartments, chambers, or other compartments.

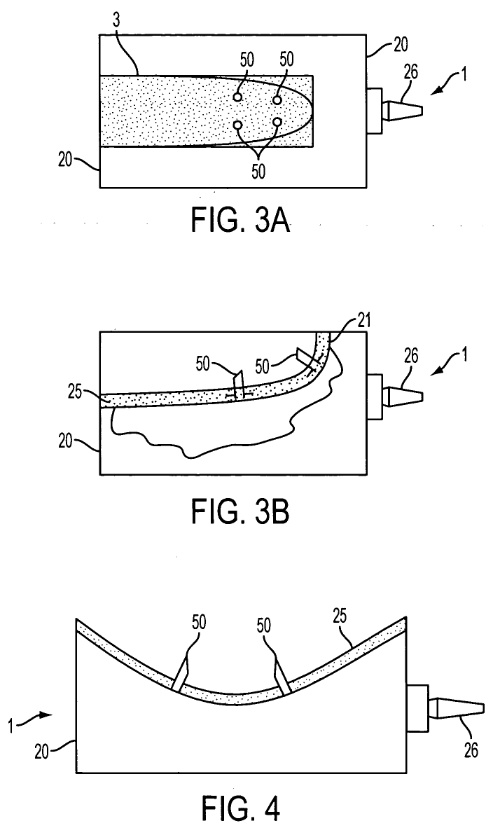

For example, referring to FIGS. 3A-3B and FIG. 4, there is shown a schematic illustration of a collection chamber 20 having a curve surface or communication surface 21. FIG. 3A is a top view illustrating a person's finger 3 resting on the collection chamber 20. Similarly, FIG. 3B is a side view illustrating a curved communication surface 21 for receiving a person's finger 3 thereon. Optionally, a manifold 25 may be utilized for mounting the cutting devices 50.

Next, FIG. 4 is a side view schematically illustrating a curved communication surface 21 for receiving body parts of a person, other than specifically a finger. Multiple curvature or planar surfaces may be implemented on at least one or more collection chambers 20.

Returning to FIGS. 1-2, sharp outlets or cutting devices 50 (i.e. hypodermic needle tips, or micro-machined needles) will be used to penetrate the skin to a set depth (i.e. about 0.1 to 15 mm or about 0.5 to 5 mm) and will be secured in the communicating surface 21 and open into the collection reservoir 20. These outlets 50 will be in an array that could have a wide range of sizes, numbers, and positions. It is envisioned that the number of outlets could range from about 1 to about 1000, with sizes up to about 8000 μm inner diameter. Outlets could optionally be secured in the manifold 25 (shown in other figures) and could penetrate the skin by hand pressure, or could be spring loaded, similar to lancets used in blood banks (or, for example, leaf or coil springs, bias elements, etc.). These outlets or cutting devices 50 will be for the aspiration of congested fluid from the congested tissue into the collection reservoir 20.

Optionally, appropriate adherence devices 22 (e.g. glue, VELCRO, tape, strap or other holding mechanism) will be used to hold the collection chamber 20 in place during treatments. For example, adhesives, straps, or micro-machined spikes along the chamber 20 or manifold 25 (shown in other figures) could also be utilized for a couple of potential actions. Bioactive agents could be adhered to the spikes and once penetrated into the skin, the agents would have an increased diffusion coefficient and diffuse into the blood stream faster. Also, these spikes could aid in adhesion by acting as a “micro-Velcro” holding the chamber 20 or other desired components in place during treatment.

In order to maintain the flow of blood and other congested fluid from the tissue 2 into the collection reservoir 20, anticoagulants and/or other thrombolytics will be used during treatments. These bioactive agents could be used systemically, locally (prior or during treatment), adhered to the outlets or cutting devices 50 and/or inside of the collection reservoir 20, or infused at the treatment site by the apparatus 1 or independent application. Tubing 28 or other desired communication channels (e.g., hose, pipe, or conduit) could also be coated with agents to prevent coagulation of the removed fluid. These bioactive agents include but are not limited to the following: anesthetics, antibiotics, antiseptics, vasodilators, anticoagulants, growth factors, and any thrombolytics. Similarly, these bioactive agents may be infused through the cutting devices 50 into the tissue during the opposing cycle of a reciprocal pump.

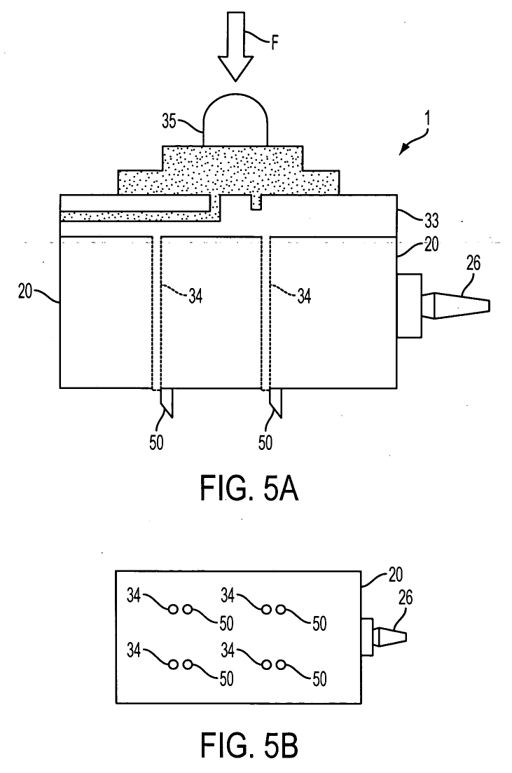

FIGS. 5A-5B schematically show a collection chamber 20 and an infusion reservoir 33 having infusion channels 34 as part of the fluid transport apparatus 1. The infusion reservoir 33 is adapted to store and deliver bioactive material or other desired material to the patient in the proximity of the cutting devices 50. During treatment or operation the bioactive material is infused or dispensed out of the infusion reservoir 33 through the infusion channels 34 and into opening of the skin where it mixes with the congested fluid that is being removed into the collection chamber 20. As one skilled in the are would appreciate various pumping mechanisms may be utilized to force the bioactive material from the infusion reservoir 33 into the skin. As shown in the FIG. 5A a primer bulb 35 is utilized, but other commercially or known pumping mechanisms may be utilized. FIG. 5B is a schematic bottom view of the collection chamber 20 that shows the reservoir channels 34 in close proximity of the cutting devices 50.

It should be appreciated, that in some embodiments the reservoir channels 34 and cutting devices 50 may be concentric with one another, i.e., one extending through the inner volume of the other respective device in some fashion. For example, the reservoir channels 34 may extend through the cutting devices 50, or conversely the cutting devices 50 may extend through the reservoir channels 34.

It should be appreciated, that in some embodiments the fluid transport apparatus and method for withdrawing fluid from a patient includes the patient having at least one target passage extending through their skin or wound, for example. As such, at least one aperture is in communication with or disposed on the collection chamber. The aperture(s) is adapted to be aligned with the target passage(s) of the patient. Consequently, the vacuum supply provides vacuum pressure to remove congested fluid from the patient via the target passage(s) and through the aperture(s) into the collection chamber. Accordingly, it is possible to practice the present invention without the cutting devices. Or alternatively, a combination with and without cutting devices may be employed in a single device.

Returning to FIGS. 1-2, the apparatus 1 will be in communication with the vacuum source 61 directly or indirectly with a vacuum tube 28 such as medical grade tubing or similar or any other fluid/air transport or channel (hose, pipe). Optionally, in series with this communication arrangement will be a “spill-off” canister 29 that will collect any volume of fluid that accumulates greater than can be held in the collection reservoir 20. Alternatively, a discharge port (not shown) may be provided to discard the excess fluid or material. This canister 29 can be of varying volumes and made of a variety of materials. To aid in the prevention of the removed fluid (blood and interstitial fluid) from flowing out of the reservoir 20 (back towards the patient or towards the vacuum source), an absorptive material (fibrous, polymer, etc.) and/or one-way valves may be placed inside the collection reservoir 20 or in-line with the “spill-off” canister 29.

Still referring to FIGS. 1-2, the vacuum source 61 can comprise, but not limited thereto a self-contained vacuum pump, which can be electric (e.g., commercially available vacuum pump), mechanical (e.g., JACKSON-PRATT drainage bulb), or existing vacuum lines that are present in hospital rooms or other facilities. In order to be more effective, the vacuum could be regulated using a desired regulator device 65 and have a pressure profile (i.e. cyclic). This regulation can be done with an electric vacuum regulator in a stand-alone unit or mechanically, by way of, but not limited to “flutter” valves in-line with the vacuum source 61 and the device/patient interface. The regulator 65 will be in between and in communication with the vacuum source 61 and collection chamber 20 at the patient interface. Alternatively, the regulator may be at or in parallel with the vacuum source 61. Performance specifications of the regulator 65 can be, but not limited to frequency, amplitude, offset, duty-cycle, pressure waveform, and treatment duration. The regulator unit 65 and/or vacuum source 61, as well as other components of the present invention, can also include an alarm or timing mechanism to signal key events in the treatment. Some key events may include but not limited to the following: volume of fluid removed, time expired, vacuum broken indicating device needs to be reattached, or removal rate of fluid and/or material. Other attributes of the device could include measuring treatment parameters; pressure sensor in any one or more chambers or tubing to determine volume of fluid removed or removal rate, skin surface or fluid temperatures, skin color via a spectrometer or equivalent device, transcutaneous oxygen level of the skin t_{c}PO₂, etc. Other key events may be identified as would be appreciated by one skilled in the art. Power can be supplied to the regulator 65 with 120V/60 Hz AC current, desired AC current, battery power supply or desired DC current.

If the device is strictly mechanical, the regulation can be done with pinch valves or similar, with the periodicity of a pressure profile incorporated with “flutter” valves or similar. It is envisioned that the flow rates that this device will be able to remove fluid from congested tissues to be between about 1 micro liter/min and about 1000 micro liter/min, or alternatively 1 micro liter/min and 10 micro liter/min. The vacuum necessary to draw these flow rates will depend on the number and size of the outlets, e.g. about 1 to about 5,000 cutting devices or alternatively about 1 to about 1,000 cutting devices. However it is thought that the relative pressure needed will be in the range of about 0 mmHg to about negative 1000 mmHg, or alternatively about negative 3 mmHg to negative 760 mmHg.

An embodiment of the present invention apparatus and method may provide an adjustable modulator, providing a pre-programmed variation (or manually on an as needed basis, or combination thereof) in duration, offset, and amplitude. This may help avoid stagnate fluid flow or fluid clots (e.g. blood clots). By enabling a variation of amplitude, offset, and duration of cycle, one could adjust for the amount of fluid that is being withdrawn from the tissue. For example, if not enough fluid is withdrawn, a stronger, longer cycle could be used, particularly at the end of a treatment cycle.

The present invention apparatus could be a modular device that would have at least one disposable module that would then be provided for off the shelf and sterile. In an embodiment, the disposable module would be the portion of the device in contact with the patient that houses the collection reservoir 20 and the manifold 25 (if provided) with the cutting outlets 50. Other potential disposable portions of the apparatus could be the “spill-off” canister 29 as well as the vacuum tube 28. The regulator 65 and potentially the vacuum pump 61 would be capital equipment that hospitals could reuse as needed for further treatments of venous congestion.

In addition, an embodiment of the present invention apparatus and method, in the mechanical mode, can also be utilized as a portable snakebite treatment therapy device.

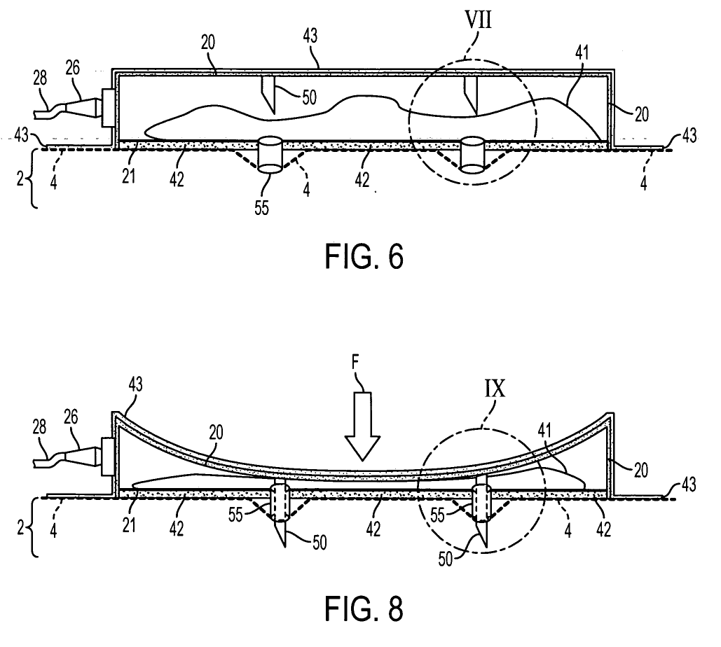

Referring to FIGS. 6-10B, an embodiment of the present invention apparatus and method comprises a collection chamber 20 that will be a reservoir for the removed congested fluid from the patient 2. As shown in FIG. 6, a connector 26 and vacuum tube 28 will communicate a negative pressure to the interior of the collection chamber 20. Inside the collection chamber 20 there will be sharp cutting devices 50 mounted to or in the vicinity of the communication surface 21 and/or manifold 25(if provided) of the chamber 20 that will be utilized for penetrating into the patient's skin 4. There may also be an absorptive material 41 inside the chamber 20 that will aid in maintaining the congested fluid that is being removed from the patient 2 to remain inside the collection chamber 22. On the communication surface 21 and/or manifold 25 the will be a sponge-like material 42 or any other suitable retainer or container in which bioactive materials will be impregnated into or stored in. Hollow communication conduits (i.e. channels, ports, outlets, pipes, tubes, or cylinders) 55 will allow communication from outside to the inside of the collection reservoir 20. The apparatus will be placed in contact with the skin 4 and will be flexible to form to the contours of the anatomy on which it is attached.

Turning to FIG. 8, dimples in the skin 4 will be created as the hollow conduits 55 (or e.g., cylinders, channels, pathway, vias) push down causing the skin 4 to become taut under tension. Optionally, an adhesive (e.g. glue, VELCRO, tape, straps) or other holding mechanism will maintain the position of the chamber 20 and/or desire portion or component of the apparatus on the skin 4. An occlusive dressing 43, such as TEGADERM, may be placed over the entire collection chamber 20 and around the peripheral skin 4 to maintain the vacuum seal. The vacuum tube connector 26 may be at least partially outside of the occlusive dressing 43 in order to connect the vacuum line 28 to the collection chamber 20

Still referring to FIG. 8, the top of the chamber(s) 20 is flexible, allowing a force as depicted as F, such as a finger force or any desired or predetermined manual or mechanical force, to depress the top allowing the cutting devices 50 to pass through the rigid (or substantially rigid) conduits 55 to penetrate into the skin 4 that is under tension. Once the cutting devices 50 penetrate into the skin 4, passive or active elastic recoil will allow the collection chamber 20 to return to its original shape. The openings in the skin 4 that have been created by the cutting devices 50 will be held open or at least partially open by the rigid conduits 55. Treatment is now ready to commence. The vacuum tube connector 26 and/or vacuum tube 28 (or the equivalent passage way as would be appreciated by one skilled in the art) will now communicate the regulated vacuum through the collection chamber 20 to the openings in the skin 4 though the hollow conduit 55 or pipe, thus allowing the congested fluid to be collected.

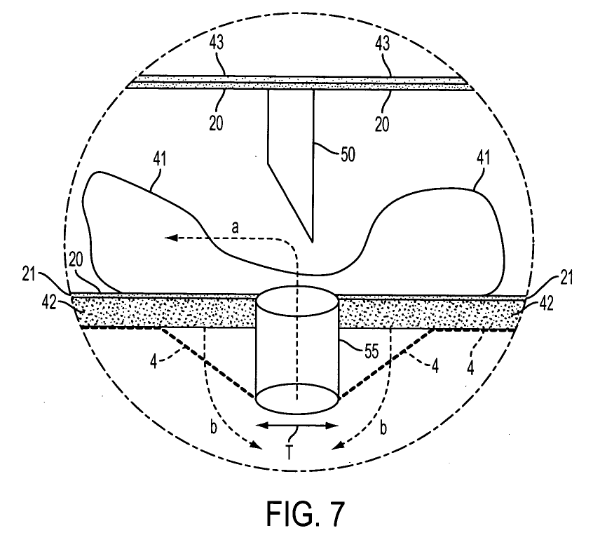

Turning to FIG. 7, there is a schematic illustration of an enlarged partial side view of the apparatus as shown in FIG. 6, during treatment or operation. The cylinder 50 dimples the skin 4 and places the skin 4 under tension as designated by T. Once the cutting devices 50 have created openings into the skin 4 the vacuum pulls the congested fluid, generally designated by the pathway a, through the conduits 55 into the collection reservoir 20 and is held in the absorptive material 41 and/or collection chamber 20. During the treatment/operation, the vacuum also pulls the bioactive material out of the sponge-like bottom layer 42 into the opening in the skin, generally designated by the pathway b, where it mixes with the congested fluid that is being removed into the reservoir 20. It should be noted that the sponge-like bottom layer can be any suitable material retainer, e.g. compartment or sack.

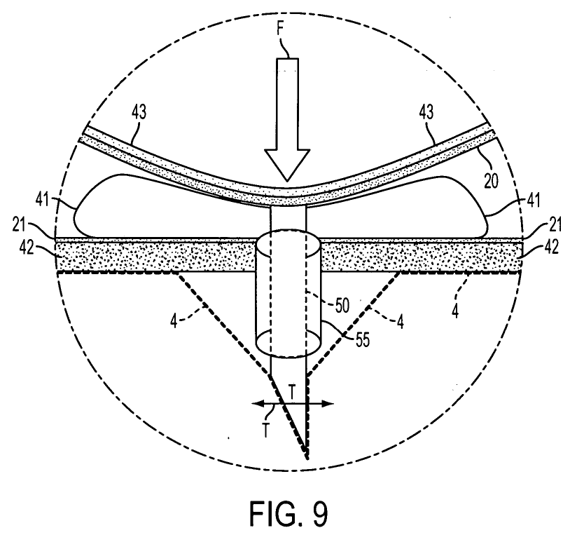

Next, turning to FIG. 9, there is a schematic illustration of an enlarged partial side view of the apparatus as shown in FIG. 8, as the cutting devices 50 penetrate the skin 4. As the top of the chamber 20 or suitable component(s) is depressed, as designated by reference F, the cutting devices 50 pass through the conduits 55 and penetrate into the skin 4 that is under tension. The cutting devices 50 are then removed from the created opening in the skin 4 by passive or active elastic recoil of the device 1 into its original configuration.

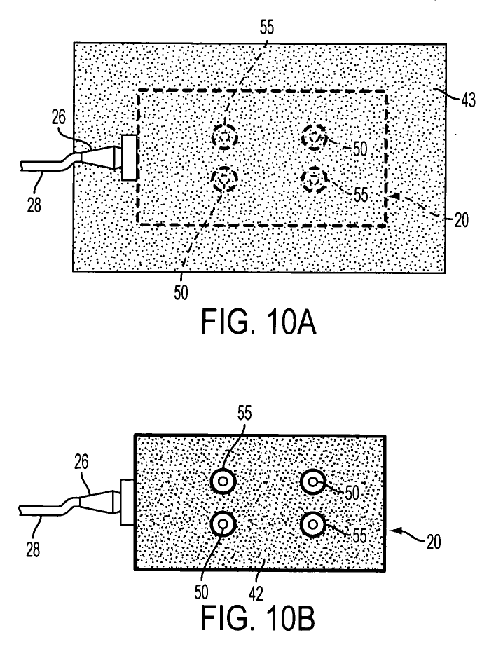

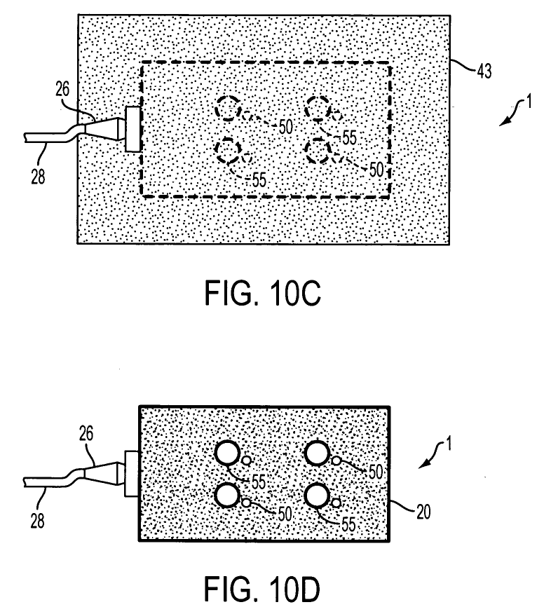

Turning to FIG. 10A, there is a schematic illustration of top plan view of the apparatus as shown in FIGS. 6 and 8. This top view shows the occlusive dressing 43 with the outline of the collection reservoir 20 and the vacuum connector 26 and/or tube 28. The cutting devices 50 and hollow cylinders 55 in concentric relation to the cutting devices 50 are shown.

Turning to FIG. 10B, there is a schematic illustration of bottom plan view of the apparatus as shown in FIGS. 6 and 8. This bottom view shows the sponge like material 42 (e.g., compartment or sack) that contains the bioactive materials. The cutting devices 50 and hollow conduits 55 in concentric relation to the cutting devices 50, and vacuum connector 26 and/or tube 28 are shown. As stated, in some embodiments the reservoir channels 34 and cutting devices 50 may be concentric with one another, i.e., one extending through the inner volume of the other respective device in some fashion. For example, the reservoir channels 34 may extend through the cutting devices 50, or conversely the cutting devices 50 may extend through the reservoir channels 34. In practice, these structures do not need to be perfectly concentric with one another, i.e., they can be aligned off center. One structure need only run through the interior of the other respective structure.

It should be appreciated, that in some embodiments the fluid transport apparatus and method for withdrawing fluid from a patient includes the patient having at least one target passage extending through their skin or wound, for example. As such, at least one conduit is in communication with or disposed on the collection chamber. The conduit(s) is adapted to be aligned with the target passage(s) of the patient. Consequently, the vacuum supply provides vacuum pressure to remove congested fluid from the patient via the target passage(s) and through the conduit(s) into the collection chamber. Accordingly, it is possible to practice the present invention without the cutting devices. Or alternatively, a combination with and without cutting devices may be implemented in a single device. It should be appreciated that the cutting devices 50 can be disposed at or near the bottom wall of the collection chamber 20 rather than the top wall as shown in FIGS. 6-10; or a combination thereof.

Turning to FIG. 10C, there is a schematic illustration of top plan(e?) view of an embodiment of the apparatus wherein the cutting devices 50 and hollow conduits 55 in juxtaposed relation to the cutting devices 50. Similarly, FIG. 10D provides a schematic illustration of bottom plan view of the apparatus wherein the cutting devices 50 and hollow conduits 55 in this juxtaposed relation to the cutting devices 50.

It should be appreciated that the cutting devices 50, conduits 55 or apertures may be disposed on or in communication with various portions or the collection chamber either directly or indirectly, as well as to the elements of the apparatus 1.

The following U.S. patents (and U.S. Patent Publication) are hereby incorporated by reference herein in their entirety:

| | |
|---|---|
| 5,582,184 | Erickson et al. |
| 4,462,405 | Ehrlich |
| 5,879,367 | Latterell et al. |
| 5,680,872 | Sesekura et al. |
| 3,742,954 | Strickland |
| 3,741,197 | Sanz et al. |
| 6,071,267 | Zamierowski |
| US 2001/0027300 A1 | Hartig et al. |
| 5,680,872 | Sesekura et al. |
| 6,340,354 | Rambin |
| 7,341 | Delluc |
| 100,210 | Stohlmann et al. |

### EXAMPLES

The following examples are intended for illustrative purposes only and are not intended to be limiting in any manner.

### Example No. 1

Referring to FIGS. 1-2, the various dimensions are normal dimensions and are not intend to be limiting in any manner. The access port 23 can be machined out or removed after the molding. Moreover, the access port may be excluded from design. The stopper to the access port 23 is required to be able to hold an air tight seal, therefore, one skilled in the art would appreciate that various types of stopper components and materials (e.g., silicone and/or rubber) would be appropriate to achieve this purpose. Also, the approximate ⅛” barbed tube connector 26 can be attached after molding, or it can be a part of the initial mold, whichever is desired. Various types of connectors, other barbed, may be utilized or the connector could be integral with the connection tube 28. The outlets 50 may to be approximately 18 gauge hypodermic needles ( approximately 1.2 mm O.D.) or other predetermined suitable sizes. In an embodiment, spring-loaded pressure released needles may be incorporated.

### Example No. 2

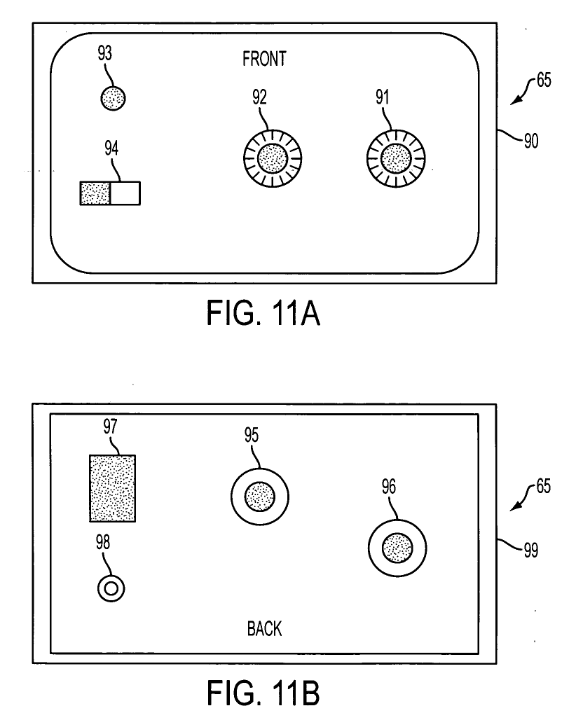

Turning to FIGS. 11A-11B, FIGS. 11A-11B show a schematic representation of an exemplary embodiment of the front and rear layouts of the control panel 90 for the regulator device 65, as well as connector ports on the back side 99, respectively. The control panel may be various control means including analog or computer interface, e.g., touch screen interface. The base vacuum level is adjusted by means of the base line pressure knob 92 and the power status is displayed on the power LED 94. Turning the knob fully counter-clockwise corresponds to a base vacuum of about 0 mmHg. Turning the knob fully clockwise corresponds to a base vacuum of about negative 10 mmHg. The Peak vacuum level is adjusted by means of the peak pressure knob 91. Turning the knob fully counter-clockwise corresponds to a peak vacuum of negative 5 mmHg. Turning the knob fully clockwise corresponds to a peak vacuum of negative 30 mmHg. The regulator device 65 can perform the base vacuum/peak vacuum cycle at two frequencies, about 0.5 Hz and 1 Hz. The operational frequency is selected by means of the frequency switch 94. If the frequency switch is set to 1 Hz, the regulator device 65 will switch between Base and Peak vacuum levels every 0.5 seconds (50% duty cycle) for a total period time of 1 second. If the frequency switch is set to 0.5 Hz, the MLRD will switch between Base and Peak vacuum levels every second (50% duty cycle) for a total period time of 2 seconds. It is envisioned that the control panel 90 can be accessed directly by using a touch screen interface or remotely by a wire or wireless communication. LED displays could also be utilized to display parameter values such as but not limited to pressure and frequency.

The connector ports on the back side 99 include vacuum source attachment 95, collection device attachment 96, power switch 97, and power adapter input 98.

### Example No. 3

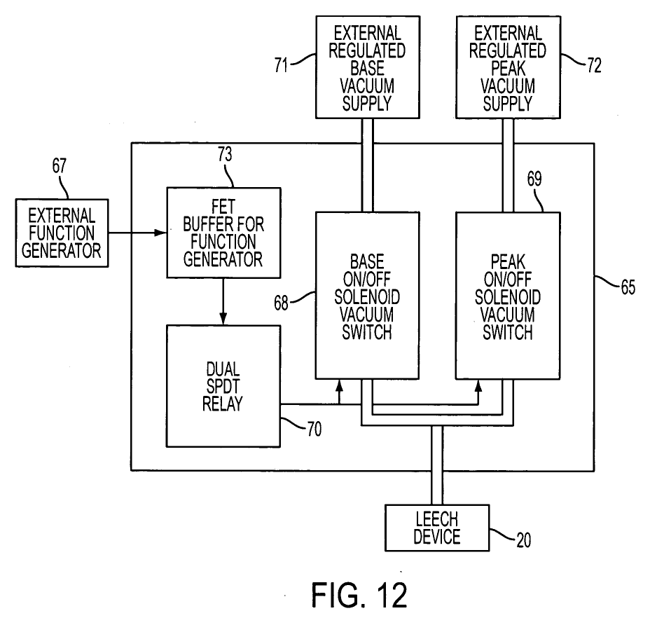

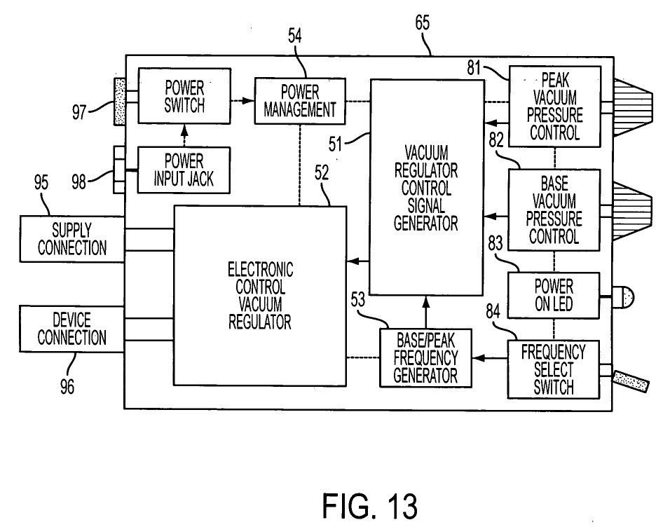

Turning to FIG. 12, FIG. 12 show circuit diagram of an exemplary embodiment of the regulator device 65 and leech device 1. The regulator device 65 is powered from an isolated AC to DC wall mount power cube at 12 Volts. An external function generator 67 and buffer are used to generate gate voltages to switch between base and peak vacuum pressures. The vacuum pressures are regulated and controlled externally by the vacuum supply modules 71, 72. Two solenoids 68, 69 are switched by a relay 70, driven by the buffered output of the function generator 67. The two solenoids control which vacuum supply, the base or peak, is delivered to the leech device 1.

### Example No. 4

Turning to FIG. 13, FIG. 13 show circuit diagram of an exemplary embodiment of the regulator device 65 comprising peak vacuum pressure module 81, base vacuum pressure control 82, power on LED 83, frequency select switch 84, power management module 54, base/peak frequency generator 53, electronic control vacuum regulator module 52, and vacuum regulator control signal generator module 51.

In conclusion, the embodiments of the present invention apparatus and method provide a number of significant advantages. For example, in the plastic surgery field there is often the need for the surgical transfer of skin flaps or the reattachment of traumatically amputated tissues. The present invention apparatus and method will aid in the removal of blood and fluid from transplanted tissues (skin flaps, ears, noses, fingers, toes, etc.) that have a compromised venous outflow of blood. By removing this congested fluid, new nutritive blood will fill the tissue and allow the tissue to live and give the body's own physiology time to reestablish adequate venous drainage.

A further advantage of the present invention apparatus and method is that it will allow for the controlled removal of fluid from the patient and an improvement over the currently utilized medicinal leeches that have very high indirect costs that are associated with them.

There are also many disadvantages to the use of leeches that include patient infection, patient fear and disgust, patient excessive scarring, patient transfusions, treatment delays during leech shipping, leeches wandering to other body parts, and many others. With a sterile off-the-shelf device, the present invention apparatus and method can also be used prophylactically, where there currently is not a treatment option.

Another advantage of the present invention apparatus and method is that during treatment/operation there are no moving parts of the apparatus.

Additional advantages of the present invention apparatus and method, but not limited thereto, are the following: reduces risk of infection; reduces indirect costs (including for example, cheaper antibiotics, minimize healthcare labor); provides monitoring, can decrease hospital stays by reducing the required number of surgical procedures in certain circumstances, reducing treatment delays normally associated with shipping leeches to the hospital sites or ineffective mechanical devices, and providing prophylactic treatments; minimize allergic response; removes blood in a controlled manner for ease of disposal; reduces scarring; and provides sterile and off-the-shelf operational units.

The present invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes which come within the meaning and range of equivalency of the claims are therefore intended to be embraced herein.

### REFERENCES

The following references are hereby incorporated by reference herein in their entirety:

1. Cottler, P. S., Skalak, T. C., “Development of a Clinically Useful Mechanical Leech Device that Promotes Flap Survival in an Animal Model of Venous Congested Skin Flaps.” Annals of Plastic Surgery, 47: 138-147, August 2001.

2. CottLer, P. S., Gampper, T. J., Rodeheaver, G. T., and Skalak, T. C., “Evaluation of Clinically Applicable Exsanguination Treatments to Alleviate Venous Congestion in an Animal Skin Flap Model.” Wound Repair and Regeneration, 7(3): 187-195, May/June 1999.

1. A fluid transport apparatus for withdrawing fluid from a patient, said fluid transport apparatus comprising:
at least one fluid collection chamber;
at least one cutting device attached to said collection chamber to penetrate the skin of the patient; and
a vacuum supply in communication with said fluid collection chamber; said vacuum supply providing a vacuum pressure to remove congested fluid from the patient through said at least one cutting device into said collection chamber.

2. The apparatus of claim 1, wherein said at least one collection chamber has a surface that is shaped to substantially adhere to the contours of a portion of the patient.

3. The apparatus of claim 1, wherein said at least one collection chamber has a surface wherein at least a portion of said surface is shaped to substantially adhere to the contours of at least a portion of the patient.

4. The apparatus of claim 1, wherein said apparatus has a surface wherein at least a portion of said surface is shaped to substantially adhere to the contours of at least a portion of the patient.

5. The apparatus of claim 1, wherein said at least one collection chamber has a plurality of surfaces wherein at least one of said plurality of said surfaces have at least one substantially curved shape.

6. The apparatus of claim 1, wherein a surface of collection chamber has a plurality of surfaces to substantially adhere to contours of a portion of the patient.

7. The apparatus of claim 1, wherein said at least one collection chamber has a volume of about 1 ml to about 100 ml.

8. The apparatus of claim 1, wherein said at least one collection chamber has a volume of about 5 ml to about 10 ml.

9. The apparatus of claim 1, wherein the number of said cutting devices range from about 1 to about 5,000.

10. The apparatus of claim 1, wherein the number of said cutting devices range from about 1 to about 1,000.

11. The apparatus of claim 1, whereby the transport rate of the fluid from the patient into said fluid collection chamber is in the range of about 10 micro liters/minute to about 100 micro liters/minute.

12. The apparatus of claim 11, whereby the number of said cutting devices that the apparatus comprises is about 1 to about 10.

13. The apparatus of claim 1, whereby the transport rate of the fluid from the patient into said fluid collection chamber is in the range of about 1 micro liters/minute to about 1,000 micro liters/minute.

14. The apparatus of claim 13, whereby the number of said cutting devices that the apparatus comprises is about 1 to about 500.

15. The apparatus of claim 1, wherein the inner diameter of said cutting devices are about 200 μm to about 840 μm

16. The apparatus of claim 1, wherein the inner diameter of said cutting devices are about 100 μm to about 200 μm

17. The apparatus of claim 1, wherein the inner diameter of said cutting devices are about 1 μm to about 8,000 μm

18. The apparatus of claim 1, further comprising:
at least one infusion chamber for storage of infusion material, said at least one infusion chamber for dispensing the infusion material in proximity of said at least cutting device to allow the infusion material to be infused from said infusion chamber to the patient

19. The apparatus of claim 18, wherein the infusion material comprises at least one bioactive agent material.

20. The apparatus of claim 19, wherein the bioactive agent material include at least one of thrombolytics, anticoagulants, anesthetics, antibiotics, antiseptics, vasodilators, anticoagulants, and growth factors.

21. The apparatus of claim 1, wherein the vacuum pressure for said apparatus provided by said vacuum supply will be in the range of about negative 3 mmHg to about 760 mmHg relative pressure.

22. The apparatus of claim 1, wherein the vacuum pressure for said apparatus provided by said vacuum supply will be in the range of about 0 mmHg to about negative 1,000 mmHg relative pressure.

23. The apparatus of claim 1, further comprising:
a regulator in communication with said vacuum supply to control vacuum pressure.

24. The apparatus of claim 23, wherein said regulator controls operating parameters, said operating parameters comprising at least one of frequency, amplitude, offset, duty-cycle, pressure waveform, and treatment duration.

25. The apparatus of claim 1, further comprising:
at least one alarm or at least one timer or both said at least one alarm and said at least one timer to signal predetermined or significant events during operation of the apparatus.

26. The apparatus of claim 25, wherein said alarm provides notification of at least one of a plurality of significant events.

27. The apparatus of claim 26, wherein said at least one of a plurality of significant events is a characteristic selected from the group consisting; volume of fluid removed, operation time expired, vacuum broken, removal rate of fluid or material, skin surface or fluid temperatures, skin color, and transcutaneous oxygen level of the skin.

28. The apparatus of claim 1, wherein said at least one cutting device penetrates the skin about 0.5 mm to about 5 mm in depth.

29. The apparatus of claim 1, wherein said at least one cutting device penetrates the skin about 0.1 mm to about 15 mm in depth.

30. A fluid transport apparatus for withdrawing fluid from a patient, whereby the patient has at least one target passage extending therein, said fluid transport apparatus comprising:
at least one fluid collection chamber;
at least one aperture in communication with said collection chamber, said at least one aperture adapted to be aligned with said at least one of the target passage of the patient; and
a vacuum supply in communication with said fluid collection chamber; said vacuum supply providing a vacuum pressure to remove congested fluid from the patient via the at least one target passage and through said at least one aperture into said collection chamber.

31. A fluid transport apparatus for withdrawing fluid from a patient, said fluid transport apparatus comprising:
at least one fluid collection chamber;
at least one cutting device disposed on said collection chamber to penetrate the skin of the patient to form at least one aperture in the skin;
at least one conduit disposed on said collection chamber, said at least one cutting device having at least a portion disposed inside said at least one conduit; and
a vacuum supply in communication with said fluid collection chamber; said vacuum supply providing a vacuum pressure to remove congested fluid from the patient through said at least one conduit into said collection chamber.

32. The apparatus of claim 31, wherein:
said at least one cutting device has a distal end furthest from said collection chamber;
said at least one conduit having a distal end furthest from said collection chamber; and
said at least one cutting device has a protrusion position whereby said distal end of said at least one cutting device is beyond the distal end of said at least one conduit.

33. The apparatus of claim 31, wherein:
said at least one cutting device has a distal end furthest from said collection chamber;
said at least one conduit having a distal end furthest from said collection chamber; and
said at least one cutting device has a protrusion position whereby said distal end of said at least one cutting device is beyond the distal end of said at least one conduit.
said at least one cutting device has a retraction position whereby said distal end of said at least one cutting device is even with or below the distal end of said at least one conduit, wherein said at least cutting device is adapted to move from said protrusion position to said retraction position.

34. The apparatus of claim 31, wherein:
said at least one cutting device has a distal end furthest from said collection chamber;
said at least one conduit having a distal end furthest from said collection chamber; and
said at least one cutting device has a protrusion position whereby said distal end of said at least one cutting device is beyond the distal end of said at least one conduit.
said at least one cutting device has a retraction position whereby said distal end of said at least one cutting device is even with or below the distal end of said at least one conduit, wherein said at least cutting device is adapted to move from said retracted position to said protrusion position and then to said retraction position.

35. The apparatus of claim 31, wherein said at least one collection chamber has a surface that is shaped to substantially adhere to the contours of a portion of the patient.

36. The apparatus of claim 31, wherein said at least one collection chamber has a surface wherein at least a portion of said surface is shaped to substantially adhere to the contours of at least a portion of the patient.

37. The apparatus of claim 31, wherein said apparatus has a surface wherein at least a portion of said surface is shaped to substantially adhere to the contours of at least a portion of the patient.

38. The apparatus of claim 31, wherein said at least one collection chamber has a plurality of surfaces wherein at least one of said plurality of said surfaces have at least one substantially curved shape.

39. The apparatus of claim 31, wherein a surface of collection chamber has a plurality of surfaces to substantially adhere to contours of a portion of the patient.

40. The apparatus of claim 31, wherein said at least one collection chamber has a volume of about 1 ml to about 100 ml.

41. The apparatus of claim 31, wherein said at least one collection chamber has a volume of about 5 ml to about 10 ml.

42. The apparatus of claim 31, wherein the number of said cutting devices range from about 1 to about 5,000.

43. The apparatus of claim 31, wherein the number of said cutting devices range from about 1 to about 1,000.

44. The apparatus of claim 31, whereby the transport rate of the fluid from the patient into said fluid collection chamber is in the range of about 10 micro liters/minute to about 100 micro liters/minute.

45. The apparatus of claim 44, whereby the number of said cutting devices that the apparatus comprises is about 1 to about 10 and the number of said conduits that the apparatus comprises is about 1 to about 10.

46. The apparatus of claim 31, whereby the transport rate of the fluid from the patient into said fluid collection chamber is in the range of about 1 micro liters/minute to about 1,000 micro liters/minute.

47. The apparatus of claim 46, whereby the number of said cutting devices that the apparatus comprises is about 1 to about 500 and the number of said conduits that the apparatus comprises is about 1 to about 500.

48. The apparatus of claim 31, wherein the inner diameter of said cutting devices are about 200 μm to about 840 μm and the inner diameter of said conduits are large enough to accommodate the outer diameter of said cutting devices.

49. The apparatus of claim 31, wherein the inner diameter of said cutting devices are about 100 μm to about 200 μm and the inner diameter of said conduits are large enough to accommodate the outer diameter of said cutting devices.

50. The apparatus of claim 31, wherein the inner diameter of said cutting devices are about 1 μm to about 8,000 μm and the inner diameter of said conduits are large enough to accommodate the outer diameter of said cutting devices.

51. The apparatus of claim 31, further comprising:
at least one infusion retainer disposed on said collection chamber for retaining infusion material, said at least one infusion retainer for dispensing the infusion material in proximity of said at least one conduit to allow the infusion material to be infused from said infusion retainer to the patient

52. The apparatus of claim 51, wherein the infusion material comprises at least one bioactive agent or material.

53. The apparatus of claim 52, wherein the bioactive agent material include at least one of thrombolytics, anticoagulants, anesthetics, antibiotics, antiseptics, vasodilators, anticoagulants, and growth factors.

54. The apparatus of claim 31, wherein the vacuum pressure for said apparatus provided by said vacuum supply will be in the range of about negative 3 mmHg to about 760 mmHg relative pressure.

55. The apparatus of claim 31, wherein the vacuum pressure for said apparatus provided by said vacuum supply will be in the range of about negative 0 mmHg to about 1,000 mmHg relative pressure.

56. The apparatus of claim 31, further comprising:
a regulator in communication with said vacuum supply to control vacuum pressure.

57. The apparatus of claim 56, wherein said regulator controls operating parameters, said operating parameters comprising at least one of frequency, amplitude, offset, duty-cycle, pressure waveform, and treatment duration.

58. The apparatus of claim 31, further comprising:
at least one alarm or at least one timer or both said at least one alarm and said at least one timer to signal predetermined or significant events during operation of the apparatus.

59. The apparatus of claim 58, wherein said alarm provides notification of at least one of a plurality of significant events.

60. The apparatus of claim 59, wherein said at least one of a plurality of significant events is a characteristic selected from the group consisting; volume of fluid removed, operation time expired, vacuum broken, removal rate of fluid or material, skin surface or fluid temperatures, skin color, and transcutaneous oxygen level of the skin.

61. The apparatus of claim 31, wherein said at least one cutting device penetrates the skin about 0.5 mm to about 5 mm in depth.

62. The apparatus of claim 31, wherein said at least one cutting device penetrates the skin about 0.1 mm to about 15 mm in depth.

63. A fluid transport apparatus for withdrawing fluid from a patient, whereby the patient has at least one target passage extending therein, said fluid transport apparatus comprising:
at least one fluid collection chamber;
at least one conduit in communication with said collection chamber, said at least one conduit adapted to be aligned with said at least one of the target passage of the patient; and
a vacuum supply in communication with said fluid collection chamber; said vacuum supply providing a vacuum pressure to remove congested fluid from the patient via the at least one target passage and through said at least one conduit into said collection chamber.

64. A fluid transport apparatus for withdrawing fluid from a patient, said fluid transport apparatus comprising:
at least one fluid collection chamber;
at least one cutting device disposed on said collection chamber to penetrate the skin of the patient to form at least one aperture in the skin;
at least one conduit disposed on said collection chamber and juxtaposed with said at least one cutting device; and
a vacuum supply in communication with said fluid collection chamber, said vacuum supply providing a vacuum pressure to remove congested fluid from the patient through said at least one conduit into said collection chamber.

65. A method of transporting fluid from a patient using a fluid transport apparatus, said method comprising:
inserting at least one cutting device into said patient wherein said at least one cutting device is disposed on said fluid collection chamber;
providing a vacuum supply in communication with said fluid collection chamber; said vacuum supply providing a vacuum pressure to remove congested fluid from the patient through said at least one cutting device into said collection chamber.

66. A method of transporting fluid from a patient using a fluid transport apparatus, said fluid transport apparatus comprising:
inserting at least one cutting device into said patient wherein said at least one cutting device is disposed on said collection chamber;
providing at least one conduit disposed on said collection chamber, said at least one cutting device having at least a portion disposed inside said at least one conduit; and
providing a vacuum supply in communication with said fluid collection chamber; said vacuum supply providing a vacuum pressure to remove congested fluid from the patient through said at least one conduit into said collection chamber.

67. A method of transporting fluid from a patient using a fluid transport apparatus, said fluid transport apparatus comprising:
inserting at least one cutting device into said patient wherein said at least one cutting device is disposed on said collection chamber;
providing at least one conduit disposed on said collection chamber, said at least one cutting device being located in close proximity of said at least one conduit; and
providing a vacuum supply in communication with said fluid collection chamber; said vacuum supply providing a vacuum pressure to remove congested fluid from the patient through said at least one conduit into said collection chamber.

68. The apparatus of any one of claims 1, 30, 31, 63, and 64, wherein said at least one collection chamber has a volume of about 0.1 ml to about 1 ml.

69. The apparatus of claim 68, wherein said at least one collection chamber has a volume of about 10 ml to about 50 ml.

70. The apparatus of claim 68, wherein said at least one collection chamber has a volume of about 50 ml to about 100 ml.

71. The apparatus of claim 68, wherein said at least one collection chamber has a volume of about 100 ml to about 1,000 ml.

72. The apparatus of any one of claims 1, 30, 31, 63, or 64, further comprising a second chamber in communication with said collection chamber.

73. The apparatus of claim 72, wherein said second chamber collects fluid received from said collection chamber.

74. The apparatus of claim 72, wherein said second chamber has a volume of about 0.1 ml to about 1,000 ml.

75. The apparatus of claim 72, wherein said second chamber has a volume of about 0.1 ml to about 1 ml.

76. The apparatus of claim 72, wherein said second chamber has a volume of about 1 ml to about 10 ml.

77. The apparatus of claim 72, wherein said second chamber has a volume of about 10 ml to about 50 ml.

78. The apparatus of claim 72, wherein said second chamber has a volume of about 50 ml to about 100 ml.

79. The apparatus of claim 72, wherein said second chamber has a volume of about 100 ml to about 1000 ml.

80. The apparatus of claim 72, whereby the transport rate of the fluid from the patient into said fluid collection chamber is in the range of about 10 micro liters/minute to about 100 micro liters/minute.

81. The apparatus of claim 72, whereby the transport rate of the fluid from the patient into said fluid collection chamber is in the range of about 1 micro liters/minute to about 1,000 micro liters/minute.

82. The method of any one of claims 65, 66, or 67, further comprising:
providing a second chamber in communication with said collection chamber, said vacuum supply providing a vacuum pressure to transfer fluid from said collection chamber to said second chamber.

83. The method of any one of claims of claim 65, 66, or 67, wherein the congested fluid is removed at a flow rate of about 10 micro liters/minute to about 100 micro liters/minute.

84. The method of any one of claims of claim 65, 66, or 67, wherein the congested fluid is removed at a flow rate of about 1 micro liters/minute to about 1,000 micro liters/minute.

85. The method of claim of claim 82, wherein the congested fluid is removed at a flow rate of about 10 micro liters/minute to about 100 micro liters/minute.

86. The method of claim of claim 82, wherein the congested fluid is removed at a flow rate of about 1 micro liters/minute to about 1,000 micro liters/minute.
